(19) United States
(12) Patent Application Publication
(10) Pub. No.: US 2022/0042991 A1
(43) Pub. Date: Feb. 10, 2022

(54) Viral Assay Method

(71) Applicant: Adaptimmune Limited, Oxfordshire (GB)

(72) Inventor: Fraser Wilkes, Oxfordshire (GB)

(21) Appl. No.: 17/275,221

(22) PCT Filed: Sep. 13, 2019
(86) PCT No.: PCT/EP2019/074556

§ 371 Date: Mar. 11, 2021

(30) Foreign Application Priority Data
Sep. 14, 2018 (GB) 1815011.0

(51) Int. Cl. G01N 33/569; G01N 33/543; G01N 21/552

(52) U.S. Cl. CPC G01N 33/56983; G01N 2333/155; G01N 21/553; G01N 33/54373

(57) ABSTRACT

This invention relates to the rapid determination of the viral titer by contacting a solution comprising viral particles, such as lentiviral particles, comprising a heterologous envelope protein, such as VSV-G, with an immobilised receptor that binds to the envelope protein, and determining the binding of the viral particles to be immobilised receptor using surface plasmon resonance (SPR).

### FIELD

The present invention relates to methods for determining the titer of viral particles in solution.

### BACKGROUND

T cells (or T lymphocytes) are found widely distributed within tissues and the tumour environment. T cells are distinguished from other lymphocytes by the presence of T cell receptors (TCRs) on the cell surface. The TCR is a multi-subunit transmembrane complex that mediates the antigen-specific activation of T cells. The TCR confers antigen specificity on the T cell, by recognising an antigen peptide ligand that is presented on the target cell by a major histocompatibility complex (MHC) molecule.

Although peptides derived from altered or mutated proteins in tumour target cells can be recognised as foreign by T cells expressing specific TCRs, many antigens on tumour cells are simply upregulated or overexpressed (so called self-antigens) and do not induce a functional T cell response. Therefore, studies have focused on identifying target tumour antigens which are expressed, or highly expressed, in the malignant but not the normal cell type. Examples of such targets include the cancer/testis (CT) antigen NY-ESO-1, which is expressed in a wide array of human cancers but shows restricted expression in normal tissues (Chen Y-T et al. Proc Natl Acad Sci USA. 1997; 94(5):1914-1918), and the MAGE-A family of CT antigens which are expressed in a very limited number of healthy tissues (Scanlan M. J. et al. Immunol Rev. 2002; 188:22-32).

Identification of such antigens has promoted the development of targeted T cell-based immunotherapy, which has the potential to provide specific and effective cancer therapy (Ho, W. Y. et al. Cancer Cell 2003; 3:1318-1328; Morris, E. C. et al. Clin. Exp. Immunol. 2003; 131:1-7; Rosenberg, S. A. Nature 2001; 411:380-384; Boon, T. and van der Bruggen P. J. Exp. Med. 1996; 183:725-729).

Robust and efficient transduction methods are required to transduce T cells with expression vectors encoding receptors, including T cell receptors (TCRs) and chimeric antigen receptors (CARs), which recognise tumour antigens. These methods would be useful for example in providing T cells for use in adoptive T cell therapy, in particular cancer therapy.

To facilitate these transduction methods, viral assay techniques are used to measure the titer of solutions of viral particles. Current viral assay techniques include immunological techniques and biological titer assays. Immunological techniques, such as p24 ELISA, measure levels of a specific protein (e.g. p24) in a solution. This protein may not be present only in intact viral particles, reducing the accuracy of the assay. Immunological techniques are also relatively slow and may take 4-5 hours to perform. Biological titer assays measure the infectivity of the viral sample on target cells. However, these assays generally take 4-9 days to perform and hence can only be performed at the end of manufacture.

Methods for the reliable and consistent determination of viral titer, in particular the titer of VSV-G pseudotyped lentiviruses, would be useful in the production of transduced mammalian cells, such as T cells for use in T cell-based immunotherapies.

### SUMMARY

The present inventors have discovered that the titer of pseudotyped viral particles can be rapidly determined in small volumes of solution by surface plasmon resonance (SPR) using immobilised receptor.

A first aspect of the invention provides a method of determining the titer of viral particles in a solution comprising;
providing a solution comprising viral particles comprising a heterologous envelope protein
contacting the solution an immobilised receptor that binds to said heterologous envelope protein, and
determining the binding of the viral particles to the immobilised receptor using surface plasmon resonance,
the amount of binding to the immobilised receptor being indicative of the titer of the viral particles in the solution.

Preferably, the heterologous envelope protein may be VSV-G and the receptor is a low density lipoprotein receptor (LDLR) protein.

In some preferred embodiments, the viral particles may be VSV-G pseudotyped lentiviral particles.

A second aspect of the invention provides the use of an immobilised LDL-receptor protein in a method according to the first aspect.

Other aspects and embodiments of the invention are described in more detail below.

### BRIEF DESCRIPTION OF THE FIGURES

FIG. 1 shows a representative sensogram following injection of a sample of VSV-G pseudotyped lentiviral particles.

FIG. 2 shows the dose response to differing concentrations of a sample of VSV-G pseudotyped lentiviral particles on different sensor chips.

### DETAILED DESCRIPTION

This invention relates to the use of surface plasmon resonance (SPR) to determine the titer of viral particles that comprise a heterologous envelope protein. A solution of viral particles is contacted with an immobilised receptor that binds to heterologous envelope protein and the extent of binding of viral particles to the receptor determined by SPR. The amount of binding as measured by SPR is indicate of the titer of the viral particles in the solution.

The titer of a solution of viral particles is the number of viral particles in a given volume. It is often expressed as viral particles, or infectious particles per mL depending on the type of assay.

A viral particle may comprise a nucleic acid vector encapsidated by one or more viral proteins and surrounded by a viral envelope of phospholipids and glycoproteins. The nucleic acid vector may comprise one or more heterologous nucleic acids, for example coding sequences for heterologous proteins, such as antigen receptors.

Viral particles are typically produced by transducing mammalian cells with a viral vector, along with one or more viral packaging and envelope vectors, as required by the specific vector system, and then culturing the transduced cells in a culture medium, such that the cells produce viral particles that are released into the medium. The viral particles may be harvested from the culture medium, optionally concentrated, and suspended in solution.

Viral titer may be measured as described herein during the production process, for example to monitor the generation and release of viral particles in the culture medium. Viral titer may also be measured as described herein after the production process, for example for research purposes or to set baselines or reference values for future production processes.

The viral vectors described herein comprise a heterologous envelope protein i.e. the viral vectors are pseudotyped viral vectors. Instead of encapsulation with envelope protein from the same virus (e.g. a lentiviral envelope protein, such as Gp120 (SU) and/or Gp41(TM)), the nucleic acid vector is encapsulated with the envelope protein from a different virus. Titer is then determined using a receptor that specifically binds the heterologous envelope protein. Methods of generating pseudotyped viral particles are well known in the art.

Preferably, viral vectors described herein are VSVg-pseudotyped. Instead of encapsulation with the endogenous envelope protein of the viral vector, the nucleic acid vector is encapsulated with the glycoprotein G of the Vesicular stomatitis virus (VSV-G) (i.e. pseudotyped). VSV-G binds to LDLR and the presence of VSV-G in the envelope of the viral particle confers a broad host cell range on the viral particles. Methods of generating VSV-G pseudotyped viral particles are well known in the art (Lo et al Curr Protoc Hum Genet. 2007 January; Chapter 12: Unit 12.7; Cronin et al Curr Gene Ther. 2005 August; 5(4): 387-398; Addgene plasmid #14888).

Suitable viral vectors may include gamma retroviral vectors, more preferably lentiviral vectors. Lentiviruses are a sub-type of retrovirus that are capable of infecting both non-dividing and actively dividing cells. Lentiviruses include Human (HIV-1 and HIV-2), simian (SIV), feline (FIV) and equine (EIV) immunodeficiency virus. A lentiviral vector is an infectious lentiviral particle that contains heterologous nucleic acid to be expressed in a target cell. Preferred lentiviral vectors may include HIV-1 vectors, for example HIV-1 vectors with the heterologous outer envelope protein, most preferably VSV-G. In some preferred embodiments, a lentiviral vector may lack the HIV-1 Vpr gene. The use of lentiviral vectors to transduce mammalian cells is well-known in the art (see for example, Merten et al Mol Ther Meth Clin Dev. (2016) 3: 16017; Tiscorna et al Nature Protocols 1, 241-245 (2006)). After transduction of a mammalian cell by a lentiviral vector particle, the single-stranded vector RNA in the particle is copied by reverse transcriptase to produce a DNA/RNA duplex. The RNA strand of the duplex is then removed by RNaseH and the resultant single-stranded DNA copied to yield a final double-stranded DNA, which is integrated into the genome of the mammalian cell.

For safety reasons, lentiviral vectors do not generally contain the genes required for replication. Suitable lentiviral vectors may be conveniently generated according to standard techniques by transfecting a packaging cell line with a transfer vector plasmid and two or more helper plasmids. Suitable packaging cells include human cells, such as HEK293 and derivatives thereof, such as HEK293T cells (Graham et al. (1977) J. Gen. Virol., 36:59-72), tsa201 cells (Heinzel et al. (1988) J. Virol., 62:3738), and NIH3T3 cells (ATCC).

Typically, the packaging cells are transiently transfected with a transfer plasmid that comprises heterologous nucleic acid for packaging into a lentiviral vector as heterologous single stranded RNA. The heterologous nucleic acid may be flanked by long terminal repeat (LTR) sequences, which facilitate integration of the transfer plasmid sequences into the host cell. The two, three, four or more helper plasmids encode the lentiviral proteins required for the generation of infectious lentiviral particles. The two or more helper plasmids may include a packaging plasmid which encodes virion proteins, such as GAG, POL, TAT, and REV; and an envelope plasmid, which encodes the heterologous envelope protein, most preferably VSV-G. In some embodiments, two packaging plasmids may be employed, a first encoding GAG and POL and a second encoding REV (Merten et al Mol Ther Methods Clin Dev. 2016; 3: 16017). Lentiviral proteins may be expressed constitutively, inducibly or transiently in the packaging cells. Following transfection with the transfer plasmid and helper plasmids, the packaging cell line generates infectious lentiviral particles that comprise the heterologous nucleic acid.

Suitable methods of preparing lentiviral vectors and transforming packaging cell lines are well-known in the art (see for example Merten et al (2016) Mol Ther Methods Clin Dev. 2016; 3: 16017; Current Protocols in Molecular Biology, Ausubel, F. M. et al. (eds.) Greene Publishing Associates, (1989); Eglitis, et al. (1985) Science 230:1395-1398; Danos and Mulligan (1988) Proc. Natl. Acad Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115).

In some preferred embodiments, heterologous nucleic acid of the viral vector may encode an antigen receptor, for example an antigen receptor that binds specifically to cancer cells. The transduction of mammalian cells with heterologous nucleic acid encoding an antigen receptor may be useful, for example in generating T cells suitable for adoptive T cell immunotherapy.

The antigen receptor encoded by the heterologous nucleic acid may be a T cell receptor (TCR). TCRs are disulphide-linked membrane anchored heterodimeric proteins, typically comprising highly variable alpha (a) and beta (β) chains expressed as a complex with invariant CD3 chain molecules. T cells expressing these type of TCRs are referred to as α:β (or αβ) T cells. A minority of T cells express an alternative TCR comprising variable gamma (γ) and delta (δ) chains and are referred to as γδ T cells. In some preferred embodiments, the heterologous nucleic acid in the transfer plasmid may encode a heterodimeric α:β T cell receptor.

Suitable TCRs bind specifically to a major histocompatibility complex (MHC) on the surface of cancer cells that displays a peptide fragment of a tumour antigen. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a cytotoxic effect on the cancer cell. Alternatively, recognition of cognate antigens by these T cell may cause the T cell to produce immunomodulatory cytokines.

The heterologous nucleic acid may encode a synthetic or artificial TCR i.e. a TCR that does not exist in nature. For example, the TCR encoded by the heterologous nucleic acid may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al J Immunol. (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs encoded by the heterologous nucleic acid may bind to cancer cells expressing one or more of the tumour antigens NY-ESO1, PRAME, alpha-fetoprotein (AFP), MAGE A4, MAGE A1, MAGE A10 and MAGE B2.

The heterologous nucleic acid may encode all the sub-units of the antigen receptor. For example, the heterologous nucleic acid may comprise a nucleotide sequence encoding a TCR α chain and a nucleotide sequence encoding a TCR β chain.

Alternatively, the antigen receptor encoded by the heterologous nucleic acid may be a chimeric antigen receptor (CAR). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85(16):5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain, or from receptors such as CD28, 41BB, or ICOS. A CAR may comprise multiple signalling domains, for example, but not limited to, CD3z-CD28-41BB or CD3z-CD28-OX40.

The heterologous envelope protein of the viral particles binds to the immobilised receptor. Suitable receptors for viral envelope proteins are well known in the art. For example, the heterologous envelope protein VSV-G binds to Low density lipoprotein receptor (LDLR; Gene ID#3949) on the mammalian cell surface protein. LDLR mediates the endocytosis of specific ligands, including VSV-G, allowing the viral particle to infect a broad host cell range. LDLR may have the reference amino acid sequence of NP_000518.1 and may be encoded by the nucleotide sequence of NM_000527.4.

A receptor, such as an LDLR protein, for use as described herein may be immobilized on a solid support. The receptor may be immobilized on the solid surface by any convenient technique, for example amine coupling. Following immobilization, excess reactive groups on the solid support may be blocked. For example, following amine coupling, excess amine groups may be blocked with ethanolamine.

In preferred embodiments, the solid support may be a SPR sensor chip. Suitable SPR sensor chips are readily available in the art (e.g. CM5 sensor chip (GE Healthcare)).

In some embodiments, the virus solution may be diluted in SPR run buffer before titer is determined. Suitable SPR run buffers are well known in the art and include 10 mm HEPES pH 7.4, 150 mm NaCl, (1×HBS-N), 1 mM Calcium Chloride. The virus solution may be diluted at least 2 fold, at least 5 fold or at least 10 fold in the buffer.

The solution of pseudotyped viral particles may be contacted with the immobilized receptor and the binding of viral particles to the immobilized receptor determined using an SPR instrument. For example, the solution of VSV-G pseudotyped viral particles may be contacted with the immobilized LDLR protein and the binding of the viral particles to the immobilized LDLR protein determined. A range of suitable SPR instruments are available from commercial suppliers and include BIAcore T200 SPR instrument (GE Healthcare, Little Chalfont, UK). Typically, binding is determined in accordance with the manufacturer's instructions and using default parameters. For example, a solution may be in contact with the immobilised receptor in the SPR instrument for 30 to 600 seconds, preferably 300 seconds at a flow rate of 1 to 100 μL/minute, preferably 104/minute.

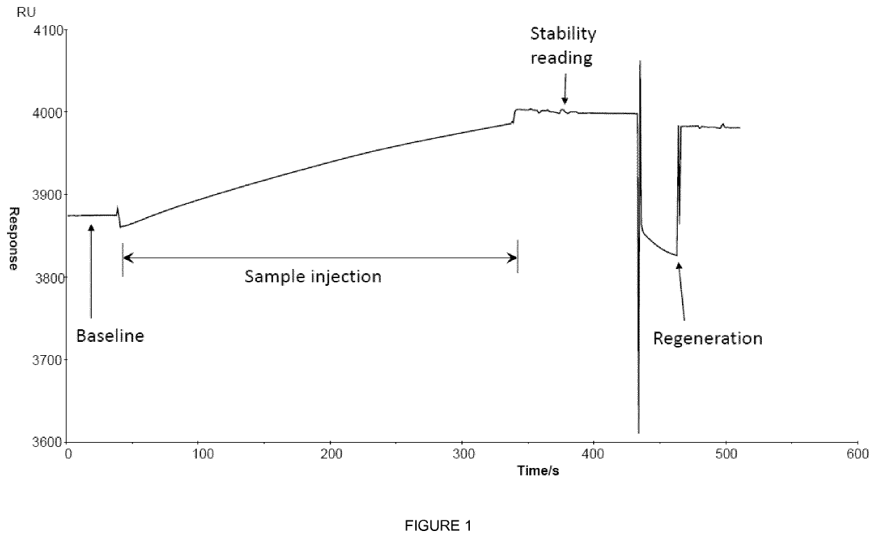

SPR involves the measurement of changes in index of refraction of the solution at surface of the solid support where binding between viral particles and immobilized receptor occurs. The changes in the refractive index are detected by the SPR instrument and recorded as RU (resonance units). RU may be monitored in real time and displayed as a sensogram (see FIG. 1). The titer of viral particles in a solution of viral particles may be determined from the RU measured for the solution. Techniques for measuring binding using SPR are well known in the art (see for example Handbook of Surface Plasmon Resonance Edition 2; Ed R Schasfort; RSC 2017)

In some embodiments, RU values determined for a solution of viral particles may be compared to reference values obtained from standard viral samples of known titer. For example, RU values obtained by SPR may be read against a standard curve to determine the titer of viral particles in the solution. A standard curve may be prepared by providing a series of standard viral solutions of known titer and determining the binding of viral particles in the standard solutions to the immobilized receptor using SPR to generate a set of reference RU values for the standard viral solutions in the series. These reference values may be used to generate a standard or calibration curve that allows the titer to be determined from the measured RU of a test solution of viral particles.

In some embodiments, a solid support, such as an SPR sensor chip, without immobilized LDLR may be used as a control. This may be useful for example in determining that the SPR instrument is working within acceptable parameters before examining test solutions of viruses.

If the titer of a solution of viral particles is found to be outside the validated range of titers for production, the titer of the solution may be adjusted, for example by dilution or concentration, in order to produce a solution within the validated range.

A titer below a threshold value may indicate a failure in the process for the production of the viral particles. If the titer of a solution of viral particles is found to be below the threshold value using a method described herein, the production process may be termination.

Optionally, following assay and optional titer adjustment, the solution of viral particles may be stored, for example by freezing at −80° C. ready for use in transducing mammalian cells.

The solution of viral particles may be used to transduce mammalian cells. Suitable methods of viral transduction are well known in the art. Suitable mammalian cells for transduction with viral particles may include primary cells, cultured cell lines, proliferating and non-proliferating cells, for example T cells, such as NK cells, B cells, dendritic cells, such as quiescent dendritic cells, stem cells, such as hematopoietic stem cells, hESCs and iPSCs, glial cells, such as microglial cells, neurons, and cancer cells.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Experimental

### Materials and Methods

All experiments were performed using a Biacore T200 (GE Healthcare) in accordance with the manufacturer's instructions.

### Reagents

Reagents were obtained from commercial suppliers as follows; soluble LDL receptor (R&D systems); Amine Coupling kit (GE healthcare); CM5 chip (GE healthcare); Sodium Acetate pH 4.5 (GE Healthcare); 10×HBS-N Run buffer (GE healthcare); NaOH (Sigma) and EDTA (Sigma).

### Chip Coupling

LDL-R was diluted in Sodium Acetate pH 4.5 to a concentration of 50 μg/mL and coupled to a CM5 chip with a target of 5000RU using standard amine coupling methods.

### Sample Injections

Samples were prepared in run buffer (1×HBS-N+1 mM Calcium Chloride) at least ten fold dilution and injected for 300 seconds at a flow rate of 104/minute.

### Surface Regeneration

A 2 step regeneration was performed: (i) 60 seconds run buffer (1×HBS-N+1 mM Calcium Chloride) 10 μL/minute then (ii) 30 seconds regeneration solution (7.5 mM NaOH, 100 mM EDTA) 50 μL/minute.

### Results

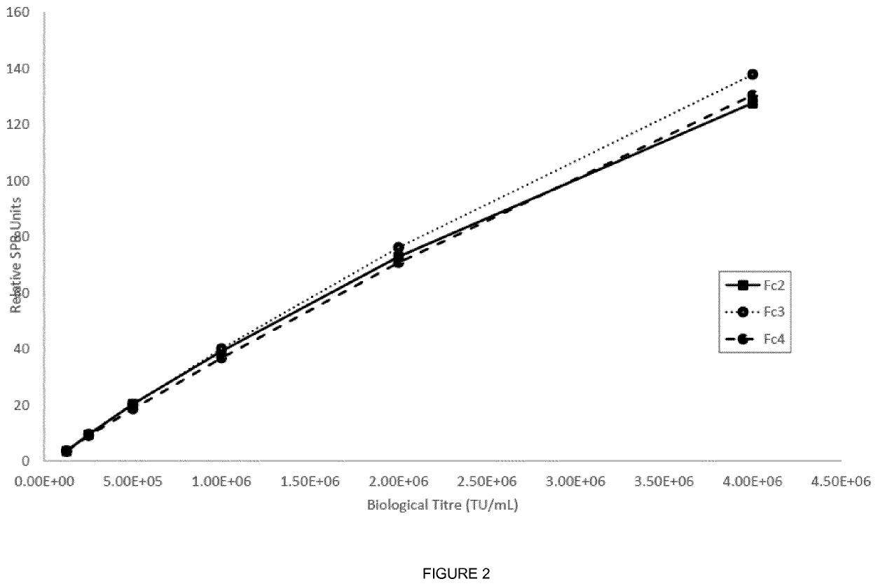

Dilutions of a sample of VSV-G pseudotyped lentiviral particles were assayed by SPR using LDL-R immobilised on three different SPR sensor chip surfaces. A representative sensogram is shown in FIG. 1. The relationship between titer and RU was found to be linear (FIG. 2). SPR therefore allows rapid determination of the titer of solutions of viral particles.

1. A method of determining the titer of viral particles in a solution comprising:
providing a solution comprising viral particles comprising a heterologous envelope protein,
contacting the solution an immobilised receptor that binds to said heterologous envelope protein, and
determining the binding of the viral particles to the immobilised receptor using surface plasmon resonance (SPR),
the amount of binding to the immobilised receptor being indicative of the titer of the viral particles in the solution.

2. The method according to claim 1 where the viral particles are lentiviral particles.

3. The method according to claim 1 wherein the heterologous envelope protein is VSV-G.

4. The method according to claim 1 wherein the receptor is a low density lipoprotein receptor (LDLR) protein.

5. The method according to claim 1 wherein the viral particles are VSV-G pseudotyped lentiviral vectors.

6. The method according to claim 1 wherein the lentiviral particles comprise a heterologous nucleic acid that encodes an antigen receptor.

7. The method according to claim 1 wherein the receptor is immobilised on a solid support.

8. The method according to claim 7 wherein the solid support is a SPR sensor chip.

9. The method according to claim 1 wherein the titer is determined by comparing the binding of the viral particles in the solution to the binding of the viral particles in one or more reference solutions of known titer.

10. Use of an immobilised immobilized LDL-receptor protein in a method according to claim 1.
